(19) United States
(12) Patent Application Publication
(10) Pub. No.: US 2025/0095821 A1
(43) Pub. Date: Mar. 20, 2025

(54) SYSTEM AND METHOD FOR PERFORMING REHABILITATION EXERCISES

(71) Applicant: NEUROTECHR3, WARREN, NJ (US)

(72) Inventors: MEE ERIKSSON, WARREN, NJ (US); QINYIN QIU, WARREN, NJ (US); AMANDA GROSS, WARREN, NJ (US); ASHLEY MONT, WARREN, NJ (US)

(73) Assignee: NEUROTECHR3, WARREN, NJ (US)

(21) Appl. No.: 18/882,967

(22) Filed: Sep. 12, 2024

Related U.S. Application Data
(60) Provisional application No. 63/538,935, filed on Sep. 18, 2023.

(51) Int. Cl. G16H 20/30; A61B 5/00; A61B 5/11; A63B 24/00

(52) U.S. Cl. CPC G16H 20/30; A61B 5/0082; A61B 5/1124; A61B 5/1128; A61B 5/702; A63B 24/0075; A61B 2505/09

(57) ABSTRACT

The system and method for performing rehabilitation exercises utilizes a support for supporting at least one body part of a user. A visual sensor visually determines a range of motion of the at least one body part, and a machine learning system is used to determine a rehabilitation exercise for performance by the user. The rehabilitation exercise is determined based on the at least one body part and the range of motion, movement speed, and ability to control the movement. The machine learning system is pre-trained with a set of rehabilitation exercises corresponding to multiple body parts and multiple ranges of motion associated therewith. The user is then guided through the determined rehabilitation exercise.

### CROSS-REFERENCE TO RELATED APPLICATIONS

This application claims the benefit of U.S. Provisional Patent Application No. 63/538,935, filed on Sep. 18, 2023.

### STATEMENT OF GOVERNMENT INTEREST

This invention was made with government support under grant no. 2101981 awarded by the National Science Foundation. The government has certain rights in the invention.

### BACKGROUND

### Field

The disclosure of the present patent application relates to physical therapy and rehabilitation, and particularly to a machine learning-based system for guiding a user through appropriate rehabilitation exercises.

### Description of Related Art

Difficulty and pain associated with the movement of joints and appendages is common, particularly due to brain injury and age. Such movement issues, if not treated, often progress to the point where the sufferer experiences debilitating pain and/or must remain at least partially immobile. Treatment of such problems may involve surgery or a physical therapy program, where a patient is instructed to perform a movement meant to stretch and strengthen the muscles and joints in the affected area. Typically, the instructions given to a patient are verbal or are in written form, often resulting in the exercises being forgotten or lost. Additionally, the descriptions of the exercises and results are self-reported back to the therapist, so it is difficult for the therapist to track exercise adherence and quality. Further, the initial evaluation of the patient for surgery or physical therapy typically requires an office visit, which may be time consuming and is often inconvenient for the patient, particularly those suffering from mobility issues.

Although the advent of telehealth may remove the travel associated with an office visit, typical telehealth systems are not effective for the types of assessments required for joint and appendage movement issues, particularly since the medical practitioner is limited only to what can be seen through the patient's camera, without any type of accurate measurements being made. Further, the results of the telehealth visit will typically be in the form of instructions, such as those described above, resulting in the same issues. Thus, a system and method for performing rehabilitation exercises solving the aforementioned problems are desired.

### SUMMARY

The system for performing rehabilitation exercises includes a support for supporting at least one body part of a user, a visual sensor, and a control system in communication with the visual sensor. The support includes a vertical support having opposed upper and lower ends, and an adapter releasably mountable to the upper end of the vertical support. The support further includes a pivoting member having an upper portion and a lower portion. The lower portion of the pivoting member is rounded and is adapted for frictional engagement with the adapter. The lower portion of the pivoting member is also adapted to pivot with respect to the adapter. A cradle is mounted on the upper portion of the pivoting member and is adapted for releasably receiving and supporting at least one body part of a user.

In one embodiment, the upper end of the vertical support has a support recess formed therein. The adapter may include a plate having opposed upper and lower ends, and a projecting member projecting downwardly from the lower end of the plate. The adapter may further have a downwardly extending adapter recess formed at least in the plate, such that the projecting member may be releasably received within the support recess. The lower portion of the pivoting member may be adapted for frictional engagement with at least a rim of the adapter recess, with the lower portion of the pivoting member being further adapted to pivot with respect to the plate of the adapter.

In an alternative embodiment of the support, the vertical support may be vertically adjustable and may include a base attached to the lower end of the vertical support and a receiver mounted on the upper end of the vertical support. The adapter may be releasably received within the receiver. Further, the base may be pivotally attached to the lower end of the vertical support and the adapter may be rotatable with respect to the receiver. The pivoting member may be releasably locked in place with respect to the adapter. Additionally, a horizontal support may be attached to the vertical support adjacent the lower end thereof, with a mount being slidably attached to the horizontal support. The mount is adapted for releasably holding the visual sensor.

In use, the control system, in combination with the visual sensor, visually determines a range of motion of the at least one body part. The control system then determines a rehabilitation exercise for performance by the user based on the at least one body part and the range of motion associated therewith. The determination of the rehabilitation exercise is performed by a machine learning system pre-trained with a set of rehabilitation exercises corresponding to multiple body parts and multiple ranges of motion associated therewith. The control system then guides the user through the determined rehabilitation exercise.

The control system, in combination with the visual sensor, may further visually determine the range of motion of the at least one body part while the user is guided through the determined rehabilitation exercise, thus allowing the previously determined range of motion of the at least one body part to be updated during and/or following performance of the rehabilitation exercise. This updated determined range of motion may then be input into the machine learning system for determination of an updated rehabilitation exercise corresponding to the user's latest level of performance. The control system may further generate a progress report for the user based on this updated determined range of motion.

The different rehabilitation exercises available to the user may include and/or involve differing degrees of difficulty based on differing levels of frictional engagement of the pivoting member with the adapter. Thus, multiple adapters may be provided to the user, in the form of a kit or the like, allowing the user to select, under guidance from the control system, a particular one of the multiple adapters having the appropriate and/or desired degree of frictional engagement with the pivoting member.

These and other features of the present subject matter will become readily apparent upon further review of the following specification.

### BRIEF DESCRIPTION OF DRAWINGS

FIG. 1 diagrammatically illustrates a system for performing rehabilitation exercises.

FIG. 2 is an exploded perspective view of a support of the system for performing rehabilitation exercises.

FIG. 3A is a partially-exploded side view of the support of FIG. 2.

FIG. 3B is a partially-exploded side view of an alternative embodiment of the support of FIG. 2.

FIG. 3C is a partially-exploded side view of another alternative embodiment of the support of FIG. 2.

FIG. 4 is a perspective view of a cradle of the support of FIG. 2.

FIG. 5 is a perspective view of a kit of different size bases for use with the system for performing rehabilitation exercises.

FIG. 6 is a bottom perspective view of the kit of different size bases of FIG. 5.

FIG. 7 is a perspective view of an alternative support for the system for performing rehabilitation exercises.

FIG. 8 is a partial perspective view of a vertically adjustable supporting member of the support of FIG. 7.

FIG. 9 is a partial perspective view of the support of FIG. 7.

FIG. 10 is a partial top perspective view of the support of FIG. 7.

FIG. 11 is a perspective view of a pivoting member and cradle of the support of FIG. 7.

FIG. 12 is a partial perspective view in section of the support of FIG. 7.

FIG. 13 is a perspective view of the support of FIG. 7, shown without an adapter, pivoting member or cradle.

FIG. 14 is a perspective view of the support of FIG. 7, shown in a partially collapsed configuration.

FIG. 15 is a perspective view of the support of FIG. 7, shown in a fully collapsed configuration.

FIG. 16 is a partial, exploded perspective view of the support of the system for performing rehabilitation exercises, illustrating interchangeability of the vertical supports of the embodiments of FIG. 1 and FIG. 7.

FIG. 17 is a partially exploded perspective view of the support of the system for performing rehabilitation exercises, illustrating interchangeability of the vertical supports of the embodiments of FIG. 1 and FIG. 7.

Similar reference characters denote corresponding features consistently throughout the attached drawings.

### DETAILED DESCRIPTION

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where systems or devices are described as having, including, or comprising specific components, or where processes or methods are described as having, including, or comprising specific process or method steps, it is contemplated that systems or devices of the present teachings can also consist essentially of, or consist of, the recited components, and that the processes or methods of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a system or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

A “subject” or a “user” herein is typically a human. In certain embodiments, a subject is a non-human mammal. Exemplary non-human mammals include laboratory, domestic, pet, sport, and stock animals, e.g., mice, cats, dogs, horses, and cows. As used herein, the term “user” refers to any single user for which use of the present systems and methods is desired. A subject or user can be considered to be in need of treatment.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Unless defined otherwise, all technical and scientific terms used herein have the same meaning as commonly understood to one of ordinary skill in the art to which the presently described subject matter pertains.

Where a range of values is provided, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

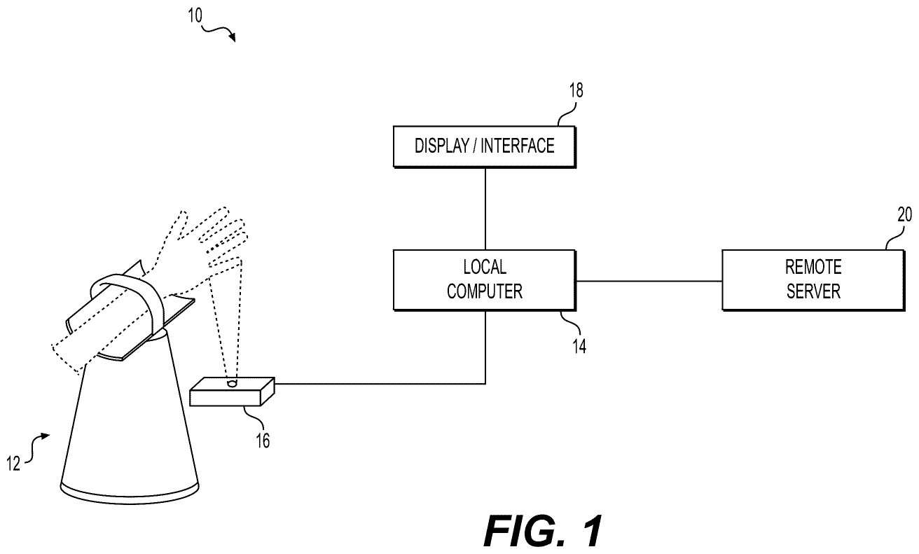

As shown in FIG. 1, the system for performing rehabilitation exercises 10 includes a support 12 for supporting at least one body part of a user, a visual sensor 16, and a control system in communication with the visual sensor 16. In FIG. 1, the control system is shown as including a local computer 14, such as a desktop computer, a laptop computer or the like, and also a remote server 20, which is connected to local computer 14 through the internet, a wide area network (WAN), a local area network (LAN) or the like. However, it should be understood that this configuration is shown in FIG. 1 for exemplary purposes only, and that any suitable type of control system may be used, including, but not limited to, only a local computer, a local controller, local control circuitry or the like, only a remote server, a remote computer, a remote computer network, remote control circuitry or the like, or combinations thereof. Further, in the non-limiting example of FIG. 1, visual sensor 16 is shown receiving the image of a user's wrist, hand and/or one or more fingers. It should be understood that the system for performing rehabilitation exercises 10 may be used for performing rehabilitation exercises on any suitable body part or combination of body parts. Although visual sensor 16 is shown as a generic video camera, it should be understood that any suitable type of camera or type of visual sensor may be used.

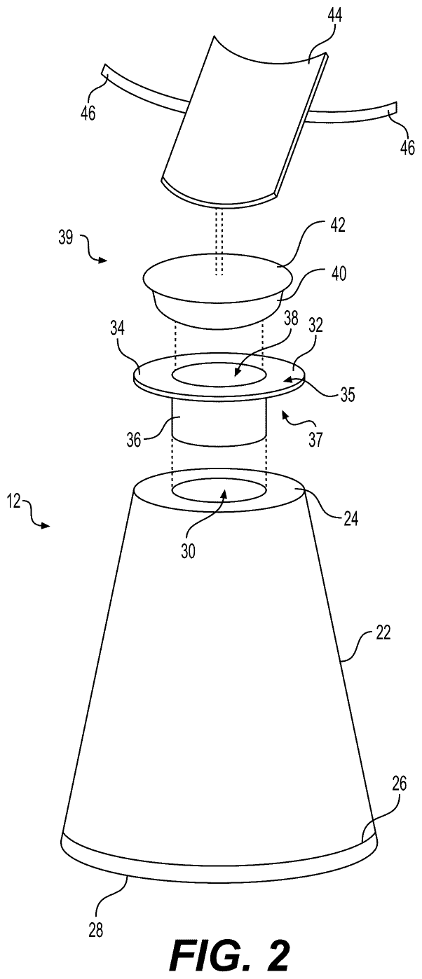

As shown in FIG. 2, the support 12 includes a vertical support, such as base 22, having opposed upper and lower ends 24, 26, respectively, with the upper end 24 having a base recess 30 formed therein. A layer of material 28, such as rubber or the like, may be secured to the lower end 26 of the base 22 for frictionally engaging a support surface, such as a desktop or the like. It should be understood that the generally frustoconical shape of base 22, and the relative dimensions associated therewith, are shown in FIG. 2 for exemplary purposes only and may be varied.

The support 12 further includes an adapter 32 having a plate 34 with opposed upper and lower ends 35, 37, respectively, and a projecting member 36 projecting downwardly from the lower end 37 of the plate 34. The adapter 32 has a downwardly extending adapter recess 38 formed at least in the plate 34, and the projecting member 36 is adapted for being releasably received within the base recess 30. It should be understood that the overall shape, configuration and relative dimensions of adapter 32 are shown in FIG. 2 for exemplary purposes only and may be varied. Similarly, although projecting member 36 may have any suitable shape and relative dimensions, projecting member 36 is configured to be securely received within base recess 30 such that adapter 32 is securely, but removably, mountable on base 22.

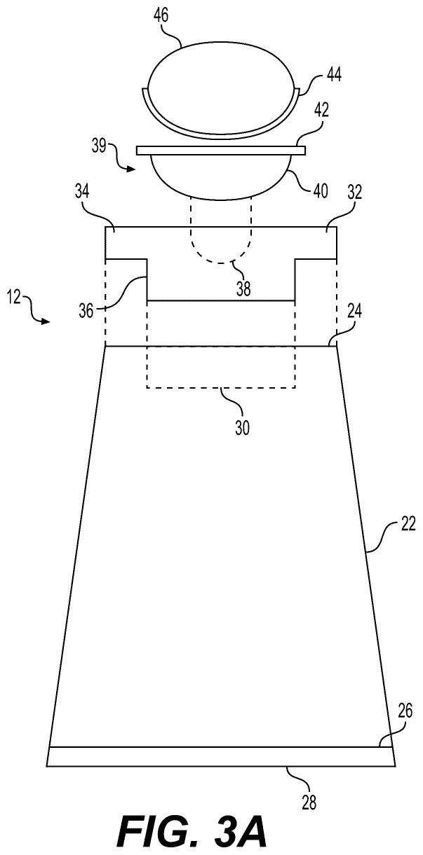

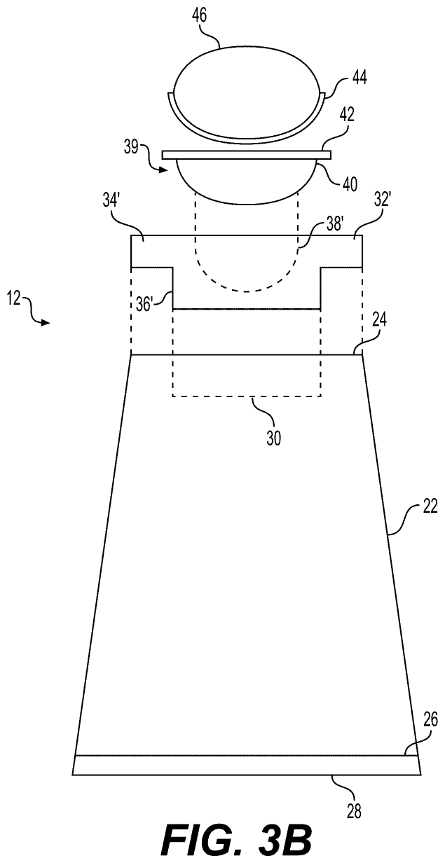

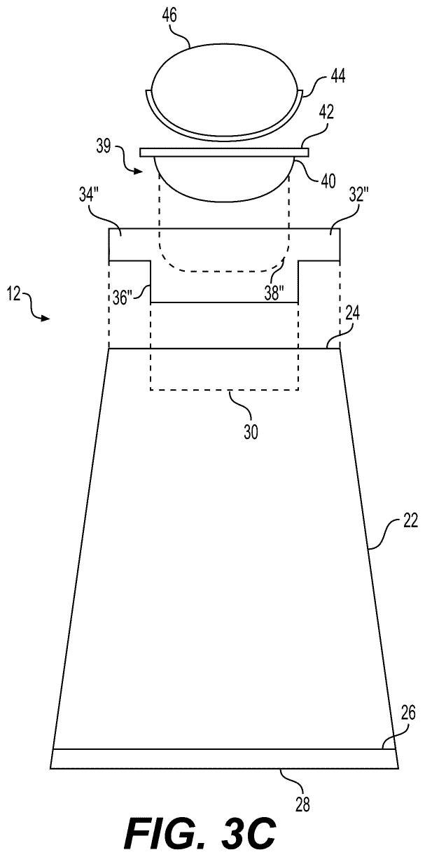

The support 12 further includes a pivoting member 39 having a lower portion 40 and an upper portion 42, with the lower portion 40 being rounded and adapted for frictional engagement with at least a rim of the adapter recess 38. The lower portion 40 of the pivoting member 39 is adapted to pivot with respect to the plate 34 of the adapter 32. As shown in FIG. 3A, in a non-limiting example where adapter recess 38 is relatively small, only a relatively small part of lower portion 40 of pivoting member 39 will be received within adapter recess 38 when pivoting member 39 sits on adapter 32. Contact between the lower portion 40 and the plate 34 of adapter 32 will only occur at the rim around the adapter recess 38. Thus, pivoting member 39 will be free to pivot with respect to adapter 32 (and base 22), but with some degree of frictional resistance.

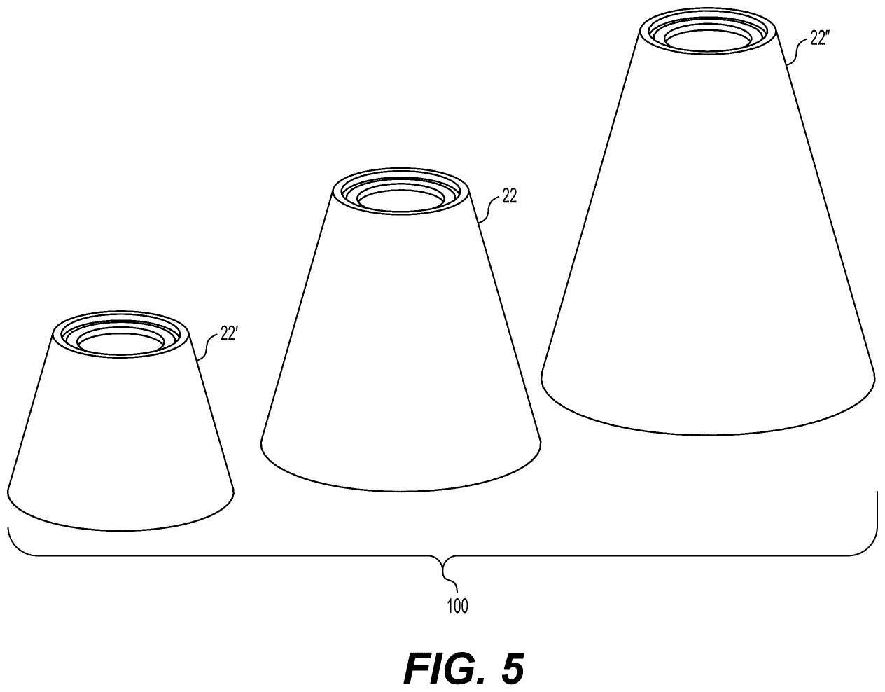

In order to provide adjustability in height for base 22, different size bases may be provided in a kit 100. In the non-limiting example of FIG. 5, three such bases 22, 22′ and 22″ are shown, although it should be understood that any number of different size bases may be contained in kit 100. The user may select the appropriate height base from bases 22, 22′ and 22″. As discussed above, it should be understood that the size, shape and relative dimensions of each base 22, 22′ and 22″ are shown in FIG. 5 for exemplary purposes only and may be varied. As a non-limiting example, instead of the regular frustoconical shape of each base 22, 22′ and 22″ shown in FIG. 5, each base 22, 22′ and 22″ (or a selected one or ones thereof) may be shaped as an oblique frustoconical base. Additionally, as shown, the base recesses may also vary in size and general configuration.

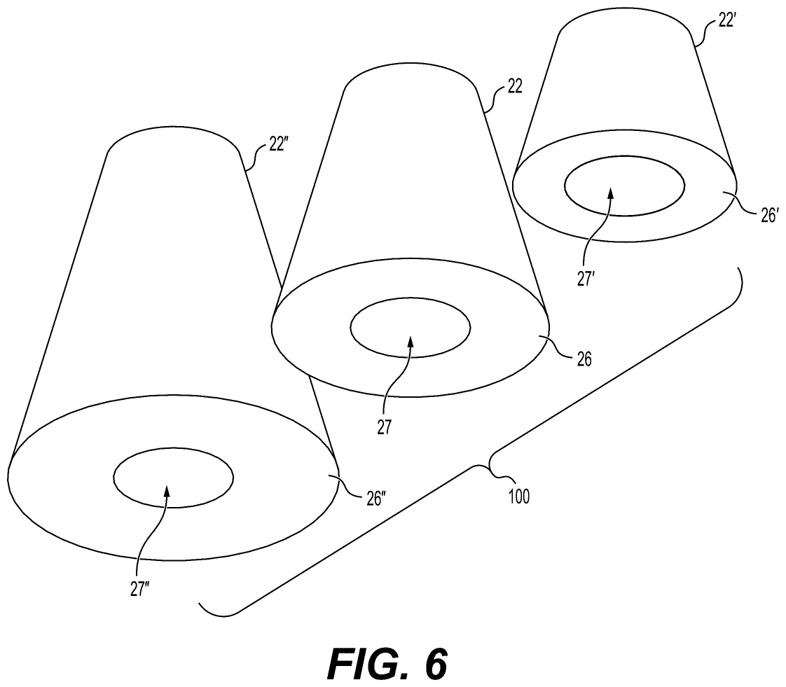

As a further alternative, as shown in FIG. 6, bases 22, 22′ and 22″ may have storage chambers 27, 27′ and 27″, respectively, formed in their respective lower ends 26, 26′ and 26″. The storage chambers 27, 27′ and 27″ may be used, as non-limiting examples, for storing pivoting members 39 and/or cradles 44 when the system for performing rehabilitation exercises 10 is not in use.

The different rehabilitation exercises available to the user may include differing degrees of difficulty based on differing levels of frictional engagement of the pivoting member 39 with the adapter 32. Thus, multiple adapters may be provided to the user, in the form of a kit or the like, allowing the user to select, under guidance from the control system, a particular one of the adapters having the appropriate degree of frictional engagement with the pivoting member 39. As a non-limiting example, adapter 32′ of FIG. 3B is similar to adapter 32 of FIG. 3A, but the adapter recess 38′ formed in the plate 34′ and the projecting member 36′ is wider than the adapter recess 38 of FIG. 3A. Since the circumference of the rim of adapter recess 38′ is larger than that of the rim of adapter recess 38, there will be a greater degree of frictional resistance when using adapter 32′ with pivoting member 39. Similarly, adapter 32″ of FIG. 3C has an even larger adapter recess 38″ formed in the plate 34′″ and the projecting member 36″, thus creating an even greater degree of frictional resistance when using adapter 32″ with pivoting member 39. In addition to varying the size and/or shape of the adapter recesses, or as an alternative thereto, adapters 32, 32′ and 32″ can be formed from different materials with different frictional coefficients. Thus, depending on the particular frictional resistance required for a particular exercise, the user may select the appropriate adapter from a kit of adapters for use with base 22 and pivoting member 39.

As non-limiting examples, adapter selection may be based on the assessed impairment level and type. For example, an adapter (or group of adapters) may be selected based on finger gross motor exercise, finger individuation exercise, wrist extension/flexion exercise, wrist radial/ulnar deviation exercise, forearm supination/pronation exercise, elbow extension/flexion exercise, shoulder extension/flexion exercise, or multi-joint coordination (or combinations thereof). As the user progresses, further adapter selection may be used to increase resistance. In order to distinguish the different adapters and their respective levels of resistance, the kit of adapters may be provided in different colors, for example. As a non-limiting example, a green adapter may be provided with an adapter recess with a diameter of 8 cm, and with the adapter made of high friction material; a blue adapter may be provided with an adapter recess with a diameter of 8 cm, and with the adapter made of low friction material; a yellow adapter may be provided with an adapter recess with a diameter of 6 cm, and with the adapter made of high friction material; a red adapter may be provided with an adapter recess with a diameter of 6 cm, and with the adapter made of low friction material; and a black adapter may be provided with an adapter recess with a diameter of 4 cm, and with the adapter made of low friction material.

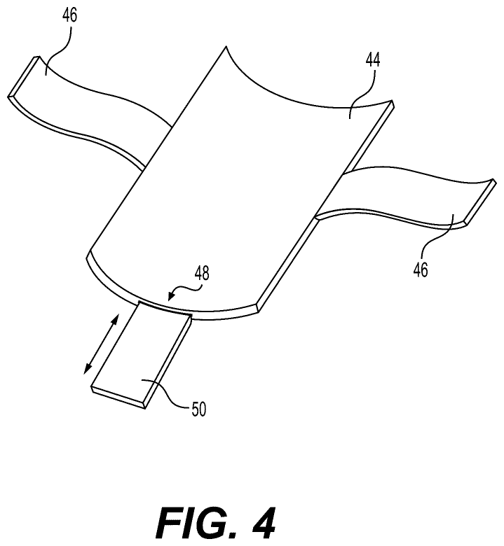

Returning to FIG. 2, the support 12 further includes a cradle 44 for releasably receiving and supporting the at least one body part of the user. The cradle 44 is mounted on the upper portion 42 of the pivoting member 39. At least one strap 46 may be secured to the cradle 44 for releasably securing the at least one body part therein. In the non-limiting example of FIG. 2, the cradle 44 is adapted for receiving the forearm of the user, however, it should be understood that the cradle 44 may have any suitable shape and relative dimensions dependent upon the particular body part(s) to be supported and the size of the user. It should be further understood that the two straps 46 shown in FIG. 2 are shown for exemplary purposes only and may be replaced by any suitable number of straps, ties or the like, and may be releasably and adjustably secured using any suitable type of releasable fixture, such as hook-and-loop fasteners, buckles, clips or the like. Further, as shown in FIG. 4, an additional wrist support 50 or the like may be slidably received within a slot 48 formed in cradle 44, allowing the additional support to be provided as required.

In use, the control system, in combination with the visual sensor 16, visually determines a range of motion of the at least one body part. In the non-limiting example of FIG. 1, analysis of the user's hand is being performed. Thus, as non-limiting examples, visual sensor 16 may be recording movements associated with finger extension/flexion, finger abduction, finger dexterity, wrist extension/flexion, forearm pronation/supination, wrist radial/ulnar deviation, hand/elbow/shoulder coordination, and combinations thereof.

As discussed above, the system for performing rehabilitation exercises 10 may be used with any suitable body part or combination of body parts, and the tabletop support 12 shown in the non-limiting example of FIG. 1 may be replaced by any suitable type of support configured for supporting other body parts. As a further non-limiting example, a chair-mounted support could be used with an appropriately positioned visual sensor 16 for recording movements associated with arm elevation, arm vertical position control, arm horizontal position control, arm orientation control, trunk stabilization, seated height support, balance control, and combinations thereof. Further, it should be understood that visual sensor 16 may be any suitable type of visual sensor 16, such as an infrared motion capture camera or the like.

The control system determines a rehabilitation exercise for performance by the user based on the at least one body part and the range of motion, speed of movement and ability of control of the movement. In the non-limiting example of FIG. 1, the range of motion is observed by visual sensor 16 which is in communication with local computer 14. The range of motion itself may be determined numerically either by local computer 14 or by remote server 20, which is in communication with local computer 14 in this non-limiting example. Further, in this non-limiting example, the determination of the rehabilitation exercise is performed by a machine learning system operating on remote server 20. The machine learning system is pre-trained with a set of rehabilitation exercises corresponding to multiple body parts and multiple ranges of motion associated therewith. Based on the selected body part, determined range of motion, speed and accuracy, the machine learning system decides which particular rehabilitation exercises are appropriate for the user at this stage of the user's physical capabilities and rehabilitation. The control system then guides the user through the determined rehabilitation exercise through a display and interface 18 associated with local computer 14. The display and interface 18 provides the user with video and may also include speakers or the like, for audio output, as well as any conventional user interface components for data entry, such as a keyboard, mouse or the like. It should be understood that the rehabilitation exercises presented to the user may be any suitable type of exercises, such as traditional movement-based exercises, movement-based games or the like.

The control system, in combination with the visual sensor 16, may further visually determine the range of motion of the at least one body part while the user is guided through the determined rehabilitation exercise, thus allowing the previously determined range of motion of the at least one body part to be updated during and/or following performance of the rehabilitation exercise. This updated determined range of motion may then be input into the machine learning system for determination of an updated rehabilitation exercise corresponding to the user's latest level of performance. The control system may further generate a progress report for the user based on this updated determined range of motion. The progress report may be presented to the user on the display and interface 18 and/or may be presented to the user's physical therapist and/or occupational therapist, who can access the user's records and/or progress reports through remote server 20.

As a non-limiting example of the typical rehabilitation process, the local computer 14, visual sensor 16, and support 12 are first set up and a connection to remote server 20 is established. The patient is positioned in front of local computer 14 and the patient's left or right hand is positioned above visual sensor 16 using support 12. Using the visual sensor 16, the system may be calibrated to the patient's range of motion of the hand. Once the particular body part and rehabilitation needs are input, the visual assessment, as described above, may be performed, including establishing the particular goals for the rehabilitation. The machine learning system determines not only the goals but the appropriate rehabilitation exercise(s) to be performed. When the appropriate rehabilitation exercise(s) are determined, instructions are provided to the patient for which adapter to use with support 12, as well as any particular adjustments which must be made to support 12. The patient is then guided through the appropriate exercise(s), which may be in the form of games or the like, while the visual sensor 16, in combination with local computer 14 and remote server 20, collects exercise/gameplay data and kinematic joint data. This data is used by the machine learning system to reassess the patient's present level and rehabilitation needs. This updated assessment is used by the machine learning system to update the rehabilitation exercise(s). The control system also generates a progress report for the patient based on the latest updates. The progress report may be presented to the patient on the display and interface 18 and/or may be presented to the patient's physical therapist and/or occupational therapist, who can access the patient's records and/or progress reports through remote server 20. Based on the updated rehabilitation plan, the process returns to the instructions and exercise(s)/game(s) presented to the patient to perform.

As discussed above, the different rehabilitation exercises available to the user may include differing degrees of difficulty based on differing levels of frictional engagement of the pivoting member 39 with the adapter 32. Thus, multiple adapters may be provided to the user, in the form of a kit or the like, allowing the user to select, under guidance from the control system, a particular one of the adapters having the appropriate degree of frictional engagement with the pivoting member 39 based on the particular rehabilitation exercise selected for the user.

As a non-limiting example, the machine learning system may first be trained using images and/or videos of various body part movements, with each image and/or video accompanied by appropriate data representing not only the particular body part and physical/medical parameters associated with the person, but also with a value indicating the quality of the movement shown. Once trained, the images and/or video recorded by the visual sensor 16, along with the kinematic data extracted from those images, is fed to the machine learning system which matches those images and/or videos to trained images/videos. The quality of the pre-trained images/videos which best match with what was recorded by visual sensor 16 then becomes the assessment value assigned to the user. This assessment value is also linked, via a lookup table or the like, with a particular rehabilitation exercise (or rehabilitation exercises) appropriate for this particular assessment.

It should be understood that any suitable type of machine learning system or systems may be used. As a non-limiting example, regression models may be trained and evaluated using the Leave-One-Out Cross Validation (LOOCV) technique, which withholds one subject's data as a testing set, while the remaining data are designated as the training set and used for feature selection, parameter tuning and training of the regression model. The subset of data features may be identified using the Correlation Feature Selection (CFS) algorithm, which attempts to maximize the correlation between the selected features and target variable while minimizing the correlation between selected features. Once the features are selected, the selected features may be fed into any suitable type of machine learning-based matching/identification system. As a further non-limiting example, the selected features may be fed into the Amazon SageMaker® Autopilot system to identify the most high-performing model that suits the dataset based on its prediction accuracy and precision. The chosen model may then be implemented in the Amazon SageMaker® platform.

As discussed above, the user's physical therapist, occupational therapist, or other service provider may remotely access the user's progress reports and/or records by logging into the remote server 20. The user's physical therapist, occupational therapist, or other service provider may be able to track real time progress, which can be used, for example, to assist with documentation required for billing and the like, and may also be used for synchronous supervision of the user and the user's progress. The user's physical therapist, occupational therapist, or other service provider may also make modifications to, and/or override, the rehabilitation plan that is generated by the machine learning system.

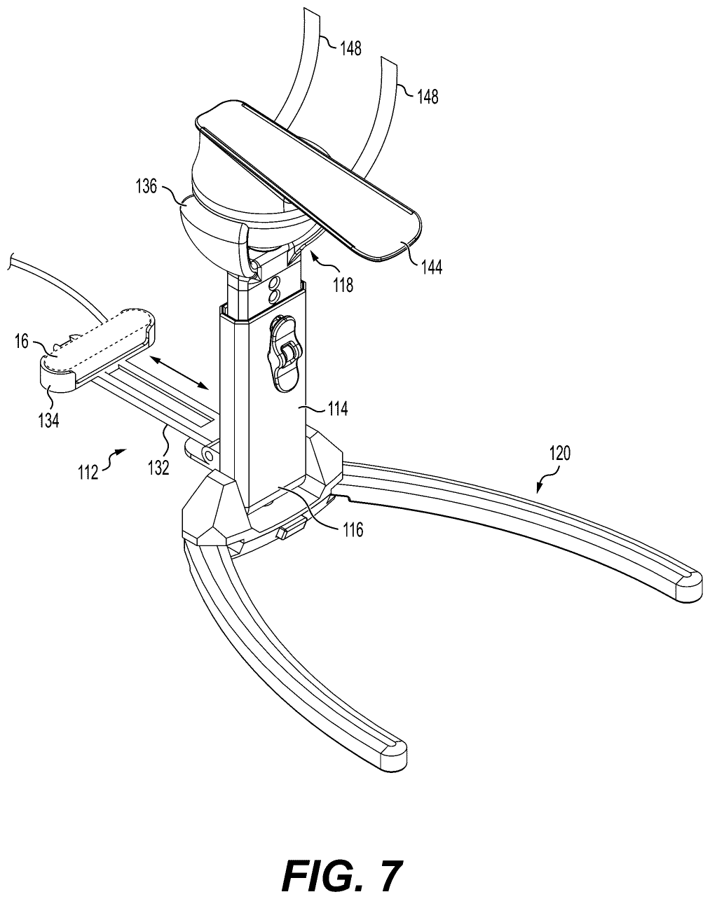

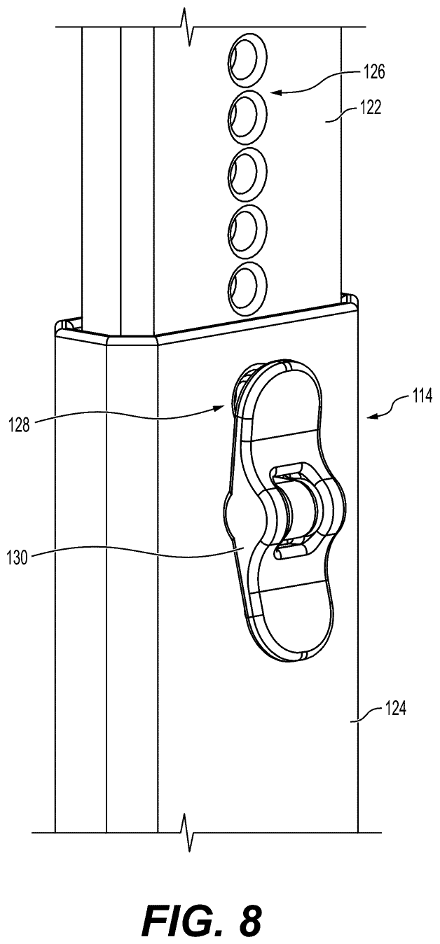

FIG. 7 illustrates an alternative support 112 for use with the system for performing rehabilitation exercises 10 described above. The support 112 includes a vertically adjustable supporting member 114 having a lower end 116 and an upper end 118. A base 120 is attached to the lower end 116 for resting on a horizontal support surface, such as a tabletop or the like. As shown in FIG. 8, the vertically adjustable supporting member 114 may be, as a non-limiting example, a telescoping bar or rod, with an inner member 122 slidable within and outer member 124. The inner member 122 may be provided with a set of vertically arrayed holes or indentations 126, as shown, for releasably receiving a peg 128 or the like to hold the inner member 122 at a desired height with respect to outer member 124. As shown in FIG. 8, peg 128 may be attached to one end of a spring-loaded lever 130 or the like, allowing the user to press down on one end of the spring-loaded lever 130 to release the peg 128 from the particular hole or indentation 126, thus allowing the vertically adjustable supporting member 114 to be telescopically adjusted. It should be understood that vertically adjustable supporting member 114 is shown for exemplary purposes only and that any suitable type of height adjustment may be used. It should be further understood that the overall shape and relative dimensions of the vertically adjustable supporting member 114 are shown for exemplary purposes only. Further, it should be understood that spring-loaded lever 130 is shown for exemplary purposes only and that any suitable type of releasable mechanism, including portions on one or both of inner member 122 and outer member 124, may be used to releasably lock the vertically adjustable supporting member 114 at a desired height.

Additionally, a horizontal support 132 may be attached at, or adjacent to, the lower end 116 of the vertically adjustable supporting member 114. The horizontal support 132 may slidably support a mount 134 for receiving the visual sensor 16. Mount 134 may be slid with respect to horizontal support 132 for horizontally adjusting the location of visual sensor 16 with respect to the body part of the user. It should be understood that the overall shape and relative dimensions of the horizontal support 132 and the mount 134 are shown for exemplary purposes only and may be varied.

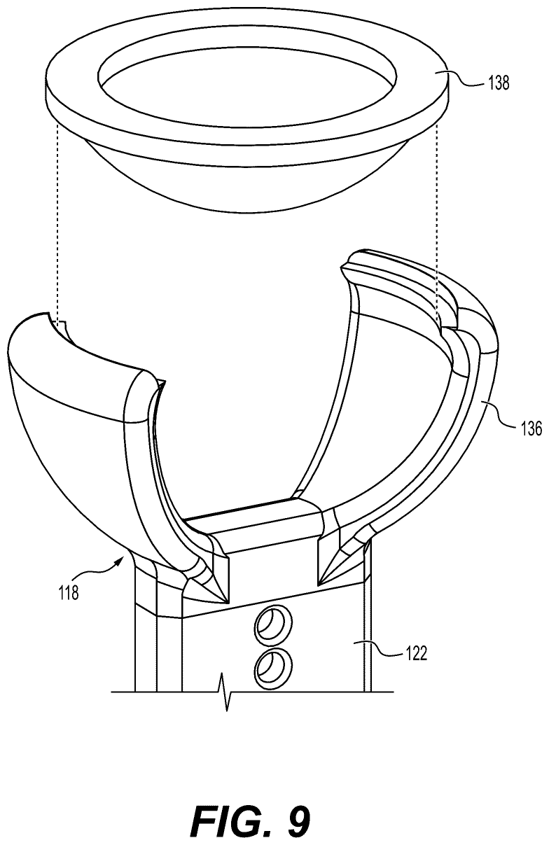

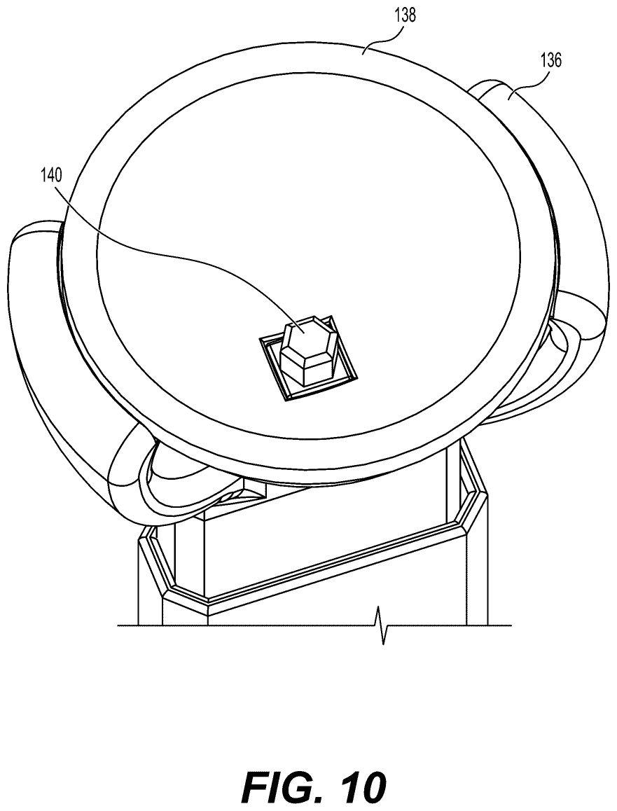

As best shown in FIG. 9, a receiver 136 is mounted on the upper end 118 of the vertically adjustable supporting member 114. The receiver 136 rotatably receives an adapter 138. Although adapter 138 serves a similar function to adapter 32 of the previous embodiment, as will be discussed in greater detail below, as shown in FIG. 10, an engaging member 140 may be removably mounted within adapter 138. It should be understood that the overall shape and relative dimensions of the receiver 136 and the adapter 138 are shown for exemplary purposes only and may be varied, however, receiver 136 and adapter 138 should be correspondingly shaped and dimensioned such that receiver 136 supports adapter 138, and such that adapter 138 is rotatable within receiver 136. Specifically, in the orientation of FIG. 9, adapter 138 is rotatable about a vertical axis through the center of the adapter 138 and receiver 136. It should be further understood that the hexagonal engaging member 140 is shown for exemplary purposes only and that engaging member 140 may have any desired shape or relative dimensions. Hexagonal member 140 may be releasably mounted on, or attached to, adapter 138 using any suitable type of releasable connection.

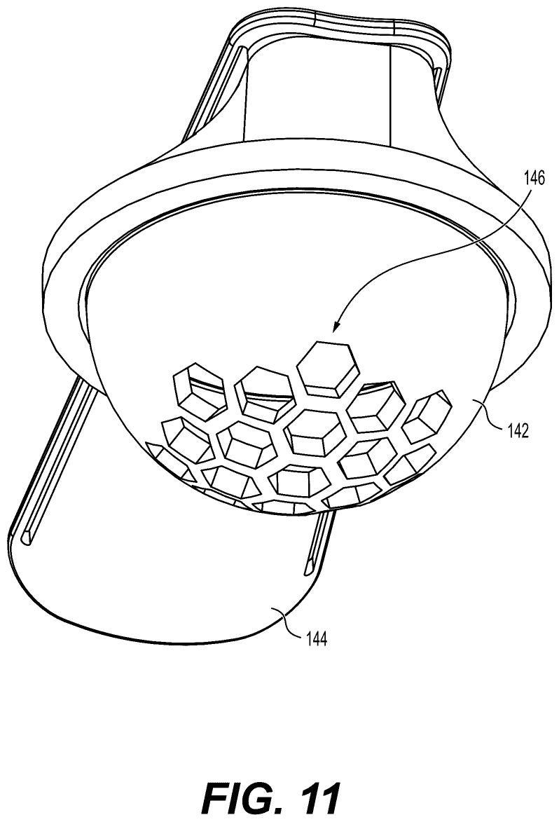

FIG. 11 shows a pivoting member 142 which is similar in function to the pivoting member 39 of the previous embodiment. The pivoting member 142 is attached to a cradle 144, similar to cradle 44 of the previous embodiment, for supporting a body part, such as the forearm of the user. As shown in FIG. 7, the cradle 144 may have straps 148 or the like, similar to straps 46 of the previous embodiment. It should be understood that the size, relative dimensions and overall configuration of the pivoting member 142 and the cradle 144 are shown for exemplary purposes only.

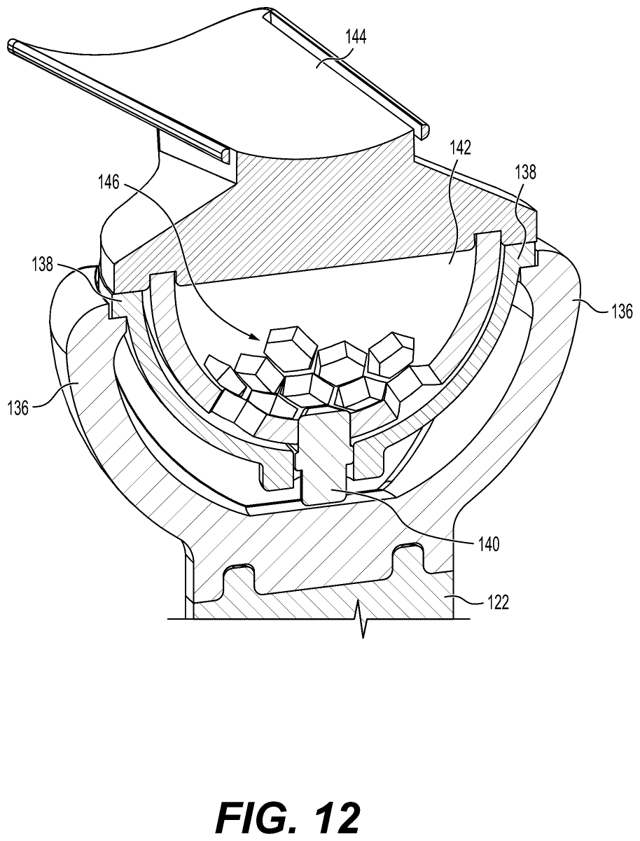

As shown in FIG. 11, pivoting member 142 may have a plurality of holes 146 formed therethrough. As shown in FIG. 12, the pivoting member 142 is received within the adapter 138. When engaging member 140 is mounted in adapter 138, the engaging member 140 enters one of the holes 146 to lock the pivoting member 142 in place with respect to adapter 138. Thus, the pivoting member 142 is free to rotate only about the same vertical axis through the center of the adapter 138 and receiver 136. However, since a plurality of holes 146 are provided, preferably spanning an angular spread of the surface of pivoting member 142, the cradle 144 can be locked at a desired angular position by choosing engagement of the engaging member 140 with an appropriate one of the holes 146. When engaging member 140 is removed from adapter 138, the pivoting member 142 may be received therein and is free to rotate with conventional three degree-of-freedom ball-and-socket movement. As discussed above with regard to the previous embodiment, the movement of pivoting member 142 with respect to adapter 138 is free in the sense of directional movement, however, there is frictional resistance between pivoting member and adapter 138. It should be understood that the hexagonal holes 146 are shown for exemplary purposes only and that holes 146 may have any desired shape and relative dimensions such that they releasably receive engaging member 140.

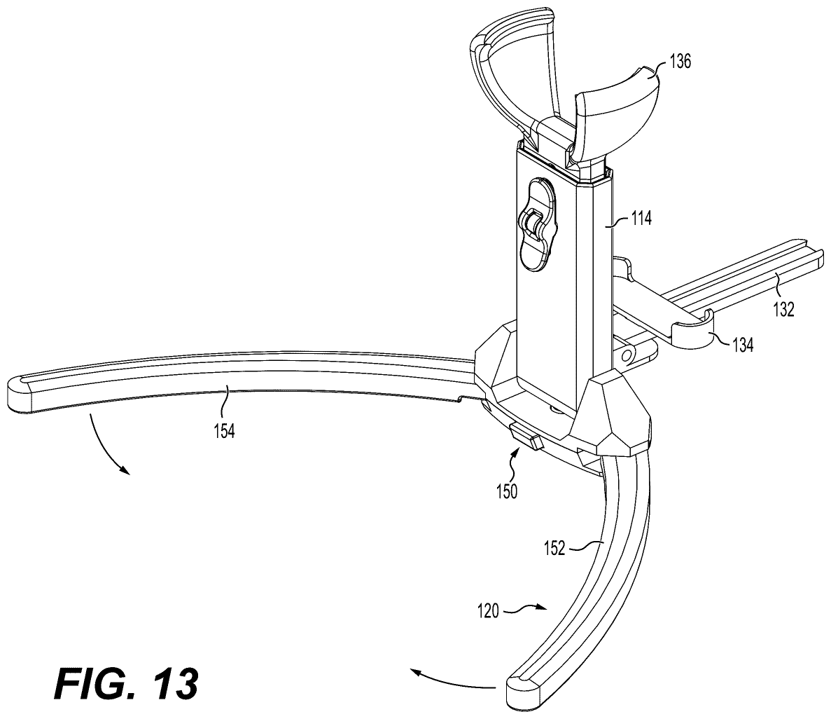

As shown in FIG. 13, when not in use, the adapter 138, pivoting member 142 and cradle 144 may be removed from the receiver 136. In FIGS. 7 and 13, base 120 is shown formed from two arcuate arms 152, 154. It should be understood that base 120 is shown in FIGS. 7 and 13 for exemplary purposes only and that the base 120 may have any desired overall configuration for supporting the support 112 on a horizontal support surface, such as a tabletop or the like. In the non-limiting example of FIGS. 7 and 13, arms 152 and 154 of base 120 may be folded together for storage and transport by pressing and unlocking button 150 or the like. It should be understood that any suitable type of release mechanism may be used to both lock the arms 152 and 154 in place during use (in the position shown in FIGS. 7 and 13) and for unlocking the arms 152 and 154 when not in use.

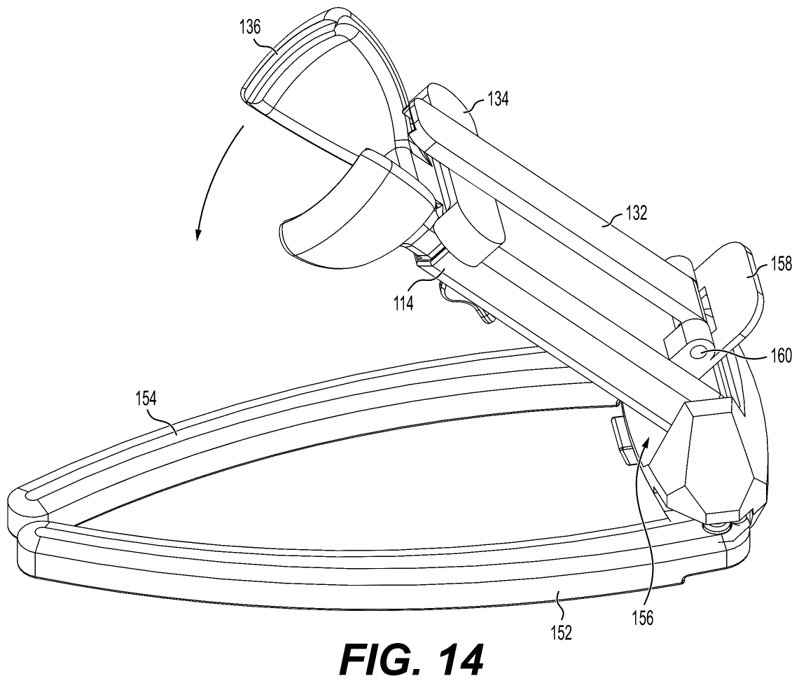

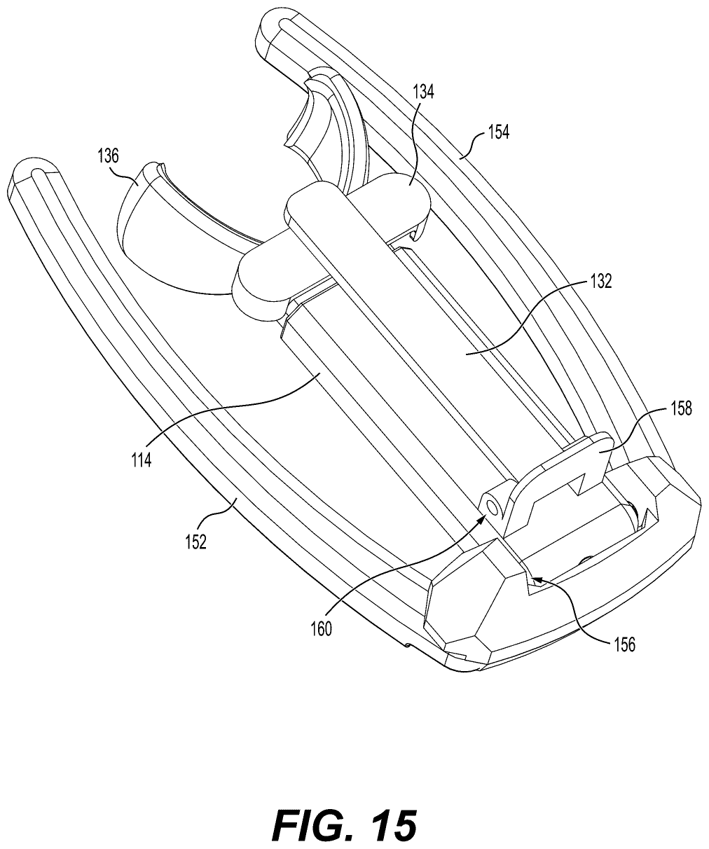

In FIG. 14, the arms 152, 154 are shown folded together after unlocking via button 150 or the like. Additionally, for purposes of storage and transport, the vertically adjustable supporting member 114 may be folded downward, toward base 120, as shown. The vertically adjustable supporting member 114 may be pivotally attached to base 120 by a hinge 156 or any other suitable type of pivotal connection. As shown in FIGS. 14 and 15, the horizontal support 132 may also be folded upward, against the vertically adjustable supporting member 114. The horizontal support 132 may be pivotally attached to the vertically adjustable supporting member 114 by a hinge 160 or any other suitable type of pivotal connection. Further, as best seen in FIG. 14, a plate 158 or other type of supporting structure may be attached to the lower end 116 of the vertically adjustable supporting member 114 to provide support for the horizontal support 132 when it is in the fully deployed position of FIG. 7.

Similar to the previous embodiment, multiple adapters 138 may be provided to the user, in the form of a kit or the like, allowing the user to select, under guidance from the control system, a particular one of the adapters having the appropriate degree of frictional engagement with the pivoting member 142. It should be understood that support 112 may be used for performing and monitoring rehabilitation exercises in a similar manner to that described above with regard to the previous embodiment.

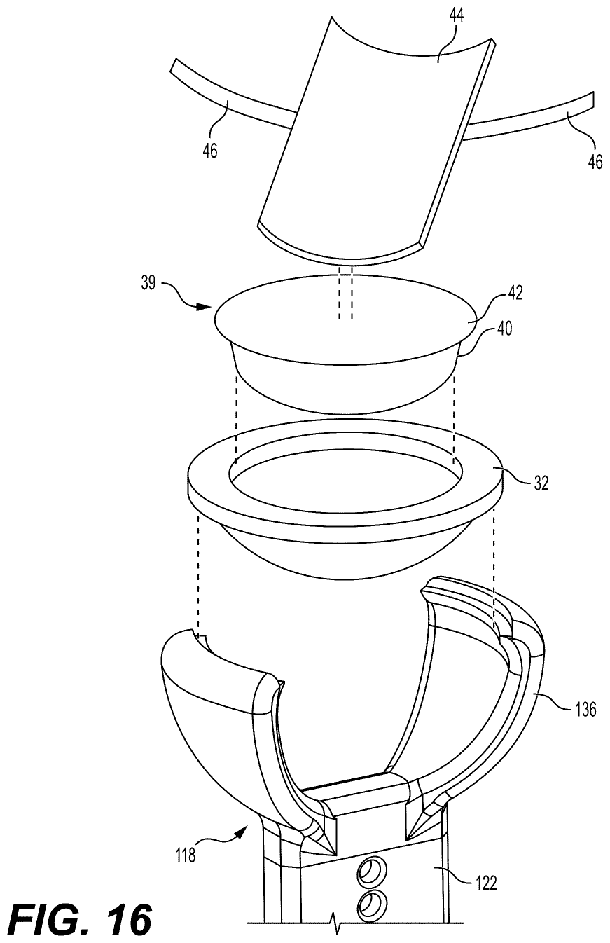

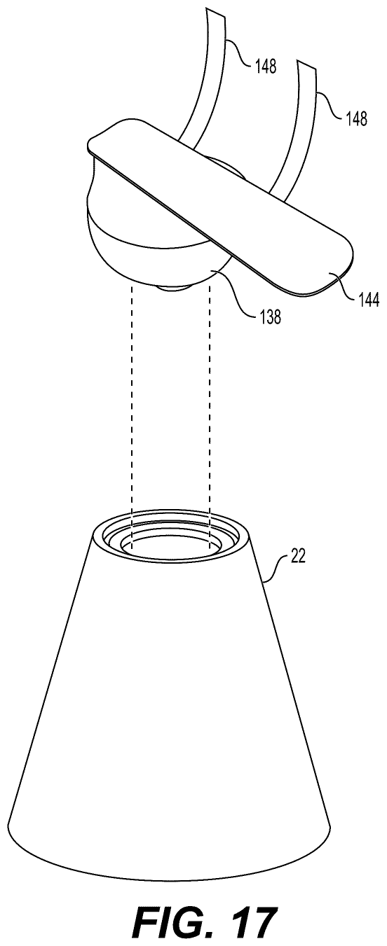

It should be understood that components of the supports 12, 112 may be interchanged. As a non-limiting example, as shown in FIG. 16, adapter 32, pivoting member 39 and/or cradle 44 of support 12 may be used in combination with vertically adjustable supporting member 114, base 120 (not shown in FIG. 16) and receiver 136 of the embodiment of FIG. 7. Similarly, as a further non-limiting example, as shown in FIG. 17, adapter 138, pivoting member 142 (not shown in FIG. 17) and/or cradle 144 of the embodiment of FIG. 7 may be used with the base 22 of the embodiment of FIG. 2.

It is to be understood that the system and method for performing rehabilitation exercises are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A system for performing rehabilitation exercises, comprising:
a support, comprising:
a vertical support having opposed upper and lower ends;
an adapter releasably mountable to the upper end of the vertical support;
a pivoting member having an upper portion and a lower portion, the lower portion of the pivoting member being rounded and being adapted for frictional engagement with the adapter, the lower portion of the pivoting member being further adapted to pivot with respect to the adapter; and
a cradle configured for releasably receiving and supporting at least one body part of a user, the cradle being mounted on the upper portion of the pivoting member;
a visual sensor; and
a control system in communication with the visual sensor, wherein the control system is configured to:
determine a range of motion of the at least one body part of the user based on visual input received from the visual sensor;
determine a rehabilitation exercise for performance by the user, wherein the rehabilitation exercise is determined based on the at least one body part of the user and the range of motion associated therewith, the determination being performed by a machine learning system pre-trained with a set of rehabilitation exercises corresponding to multiple body parts and multiple ranges of motion associated therewith; and
guide the user through the determined rehabilitation exercise.

2. The system for performing rehabilitation exercises as recited in claim 1, wherein the upper end of the vertical support has a support recess formed therein, and wherein the adapter comprises:
a plate having opposed upper and lower ends; and
a projecting member projecting downwardly from the lower end of the plate, wherein the adapter has a downwardly extending adapter recess formed at least in the plate, wherein the projecting member is adapted for being releasably received within the support recess, and wherein the lower portion of the pivoting member is adapted for frictional engagement with at least a rim of the adapter recess, the lower portion of the pivoting member being further adapted to pivot with respect to the plate of the adapter.

3. The system for performing rehabilitation exercises as recited in claim 1, further comprising a layer of material secured to the lower end of the vertical support adapted for frictionally engaging a support surface.

4. The system for performing rehabilitation exercises as recited in claim 1, wherein the support further comprises at least one strap configured for releasably securing the at least one body part in the cradle.

5. The system for performing rehabilitation exercises as recited in claim 1, wherein the control system is further configured to determine the range of motion of the at least one body part as the user is guided through the determined rehabilitation exercise.

6. The system for performing rehabilitation exercises as recited in claim 5, wherein the control system is further configured to update the determined range of motion of the at least one body part for an updated determination of the rehabilitation exercise.

7. The system for performing rehabilitation exercises as recited in claim 6, wherein the control system is further configured to generate a progress report based on the updated determined range of motion.

8. The system for performing rehabilitation exercises as recited in claim 1, wherein the vertical support is vertically adjustable.

9. The system for performing rehabilitation exercises as recited in claim 1, further comprising:
a base attached to the lower end of the vertical support; and
a receiver mounted on the upper end of the vertical support,
wherein the adapter is releasably received within the receiver.

10. The system for performing rehabilitation exercises as recited in claim 9, wherein the base is pivotally attached to the lower end of the vertical support.

11. The system for performing rehabilitation exercises as recited in claim 9, wherein the adapter is rotatable with respect to the receiver.

12. The system for performing rehabilitation exercises as recited in claim 11, wherein the pivoting member may be releasably locked in place with respect to the adapter.

13. The system for performing rehabilitation exercises as recited in claim 12, wherein an engaging member is releasably mounted to the adapter, the engaging member being adapted to be removably received within at least one hole formed through the pivoting member to selectively lock the pivoting member in place with respect to the adapter.

14. The system for performing rehabilitation exercises as recited in claim 1, further comprising:
a horizontal support attached to the vertical support adjacent the lower end thereof; and
a mount slidably attached to the horizontal support, the mount being adapted for releasably holding the visual sensor.

15. The system for performing rehabilitation exercises as recited in claim 14, wherein the horizontal support is pivotally attached to the vertical support.

16. A method for performing rehabilitation exercises, comprising the steps of:
a) supporting at least one body part of a user on a support, wherein the support comprises:
a vertical support having opposed upper and lower ends;
an adapter releasably mountable to the upper end of the vertical support;
a pivoting member having an upper portion and a lower portion, the lower portion of the pivoting member being rounded and being adapted for frictional engagement with the adapter, the lower portion of the pivoting member being further adapted to pivot with respect to the adapter; and
a cradle for releasably receiving and supporting the at least one body part of a user, the cradle being mounted on the upper portion of the pivoting member;
b) visually determining a range of motion of the at least one body part of the user with a visual sensor;
c) determining a rehabilitation exercise for performance by the user, wherein the rehabilitation exercise is determined based on the at least one body part of the user and the range of motion associated therewith, the determination being performed by a machine learning system pre-trained with a set of rehabilitation exercises corresponding to multiple body parts and multiple ranges of motion associated therewith; and
d) guiding the user through the determined rehabilitation exercise.

17. The method for performing rehabilitation exercises as recited in claim 16, further comprising the step of visually determining the range of motion of the at least one body part as the user is guided through the determined rehabilitation exercise.

18. The method for performing rehabilitation exercises as recited in claim 17, further comprising the step of updating the determined range of motion of the at least one body part and returning to step c).

19. The method for performing rehabilitation exercises as recited in claim 18, further comprising the step of generating a progress report based on the updated determined range of motion.

20. The method for performing rehabilitation exercises as recited in claim 16, further comprising the step of selecting the adapter from a plurality of adapters each having a different degree of frictional engagement with the pivoting member.
